(19) United States
(12) Patent Application Publication
(10) Pub. No.: US 2016/0304830 A1
(43) Pub. Date: Oct. 20, 2016

(54) FUNGAL TREATMENT TO ENHANCE EXTRACTABLE RUBBER YIELD FROM PLANTS

(71) Applicant: Ohio State Innovation Foundation, Columbus, OH (US)

(72) Inventors: Katrina Cornish, Wooster, OH (US); Naeem Ali, Islamabad (PK); Shomaila Sikander, Wooster, OH (US)

(21) Appl. No.: 15/098,999

(22) Filed: Apr. 14, 2016

Related U.S. Application Data
(60) Provisional application No. 62/147,081, filed on Apr. 14, 2015.
(60) Provisional application No. 62/168,454, filed on May 29, 2015.

(51) Int. Cl. C12N 1/14; C07G 1/00; C12N 9/24; C12P 19/02; C12N 9/42; C08L 7/00; C12P 19/12

(52) U.S. Cl. CPC C12N 1/14; C08L 7/00; C07G 1/00; C12P 19/12; C12P 19/02; C12N 9/2437; C12N 9/2402; C12N 9/2482; C12Y 302/01; C12Y 302/01004; C12Y 302/01007; C12Y 302/01008

(57) ABSTRACT

Materials and methods to enhance extractable natural rubber yields from plants are disclosed. The materials and methods of the present invention can be used with minimal capital investment, and can provide new agricultural, manufacturing, sales, and transport jobs for local economies. The materials and methods described herein can also be used in conjunction with presently known natural rubber extraction methods to enhance extractable natural rubber yields. Also describe herein are methods for producing a crude enzyme extract for use in rubber extraction methods of the present invention.

### CROSS-REFERENCE TO RELATED APPLICATIONS

This application claims priority to U.S. Provisional Application No. 62/168,454, filed May 29, 2015 and U.S. Provisional Application No. 62/147,081, filed Apr. 14, 2015, the entire disclosures of which are expressly incorporated herein by reference for all purposes.

### BACKGROUND OF THE INVENTION

Natural rubber cis-polyisoprene, with a molecular mass of 10,000 to 10 million g/mol, is one of the world's most important industrial raw materials. Plant-derived natural rubber is used in over 50,000 different products. There is a strong reliance on natural rubber for the manufacture of these products, primarily because synthetic alternatives cannot match the high-performance properties of natural rubber required for many applications. High performance synthetic rubbers also tend to be prohibitively expensive.

Presently, the sole commercial source of natural rubber is the para rubber tree Hevea brasiliensis. Natural rubber is obtained from Hevea by tapping the tree, collecting its latex, and coagulating its latex to obtain rubber. Most Hevea-derived natural rubber originates in Indonesia, Malaysia, Thailand, India, and tropical western Africa. These natural rubber sources are under intense threat from potential diseases and blight due to the genetic similarity of the clonally-propagated rubber trees. Furthermore, the crop is limited by a restricted geographic growth area and labor intensive harvesting methods.

Other sources, such as Taraxacum kok-saghys (also known as Buckeye Gold, rubber dandelion, Kazak dandelion, Russian dandelion, and TKS) and Parthenium argentatum (guayule) are being explored as alternative sources for natural rubber. Various methods for recovering solid natural rubber from these and other rubber-bearing plants are known in the art. These processes are generally based on wet-milling and/or solvent extraction, while some extract natural rubber utilizing dry milling processes. However, present methods are unable to fully separate solid natural rubber from non-rubber plant matter, thereby limiting extractable solid rubber yields. Furthermore, these methods are often highly-mechanized, utilizing expensive, specialized equipment. It would be advantageous to increase the yield of natural rubber from plants utilizing a mechanically simple process.

### SUMMARY OF THE INVENTION

Materials and methods to enhance extractable natural rubber yields from plants are disclosed. The materials and methods of the present invention can be used with minimal capital investment, and can provide new agricultural, manufacturing, sales, and transport jobs for local economies. The materials and methods described herein can also be used in conjunction with presently known natural rubber extraction methods to enhance extractable natural rubber yields. Also describe herein are methods for producing a crude enzyme extract for use in rubber extraction methods of the present invention.

In a particular embodiment disclosed herein is a method for enhancing yield of natural rubber from plant material, comprising the steps of: a) providing an aqueous solution or slurry comprising plant material, wherein the plant material comprises natural rubber; b) inoculating the aqueous solution or slurry with an effective amount of live Thermomyces lanuginosus or one or more derivatives thereof; c) incubating the aqueous solution or slurry with the effective amount of live Thermomyces lanuginosus or one or more derivatives thereof; and d) recovering natural rubber from the aqueous solution or slurry following the incubation.

The plant material can be chopped, ground, homogenized, or a combination thereof; subjected to an alkaline pretreatment prior to the inoculation; boiled one or more times to extract inulin; or any combination of these prior to inoculation.

The plant material of any one of the methods herein is a plant or a part of a plant selected from the group of plants consisting of: rubber dandelion (Taraxacum kok-saghyz); guayule (Parthenium argentatum); rubber tree (Hevea brasiliensis); gopher plant (Euphorbia lathyris); mariola (Parthenium incanum); rabbitbrush (Chrysothanmus nauseosus); candelilla (Pedilanthus macrocarpus); Madagascar rubber vine (Cryptostegia grandiflora); milkweeds (Asclepias spp.); goldenrods (Solidago spp.); Scorzonera tau-saghyz; mountain mint (Pycnanthemum incanum); American germander (Teucreum canadense); tall bellflower (Campanula americana); Palaquium gutta; rubber fig (Ficus elastic); Indian banyan (Ficus benghalensis); Panama rubber tree (Castilla elastic); prickly lettuce (Lactuca seriola); crisphead lettuce (Lactuca sativa); pale Indian plantain (Cacalia atriplicifolia); Canada germander (Teucrium canadense); sunflower (Helianthus annus); painted spurge (Euphorbia heterophylla); Euphorbia lactiflua; Euphorbia characias; jackfruit (Artocarpus heterophyllus); and a combination thereof. In a particular embodiment, the method of claim 1, wherein the plant material is a plant or a root of Taraxacum kok-saghyz.

In certain embodiments, the effective amount is an amount of live Thermomyces lanuginosus or one or more derivatives thereof capable of disrupting bonds between a bagasse fraction of the plant material and a natural rubber fraction of the plant material, thereby enhancing yield of natural rubber from the plant material relative to a method wherein live Thermomyces lanuginosus or one or more derivatives thereof are not employed.

In other embodiments, the one or more derivatives of the live Thermomyces lanuginosus comprises a crude enzyme extract, one or more purified enzymes isolated from a crude enzyme extract, or a combination thereof. In certain embodiments the live Thermomyces lanuginosus is Thermomyces lanuginosus isolate STm, the partial genome sequence of which is accessible as GenBank accession number KJ432867.1.

In other embodiments, the incubation occurs at a temperature of about 40° C. to about 90° C., and occurs for a duration of at least 12 hours.

In certain embodiments, the natural rubber is recovered by a method selected from the group consisting of: flotation and skimming; solvent extraction; centrifugation; and a combination thereof.

In yet other embodiments, methods for extracting natural rubber further comprise recovering fermentable sugars, lignin, or a combination thereof from the aqueous solution or slurry.

In another particular embodiment described herein is a method for purifying natural rubber comprising the steps of: a) providing a natural rubber; and b) contacting the natural rubber with an effective amount of Thermomyces lanuginosus or one or more derivatives thereof, thereby purifying the natural rubber. The natural rubber can be provided in an aqueous solution, wherein the rubber has a water content of at least 10%. For purification, the effective amount is an amount of live Thermomyces lanuginosus or one or more derivatives thereof capable dissolving at least a fraction of solids entrained in the natural rubber.

In a particular embodiment described herein is a method for lignocellulosic biomass saccharification comprising the steps of: a) providing an aqueous solution or slurry comprising lignocellulosic plant material; b) inoculating the aqueous solution or slurry with an effective amount of Thermomyces lanuginosus or one or more derivatives thereof; c) incubating the aqueous solution or slurry with the effective amount of Thermomyces lanuginosus or one or more derivatives thereof; and d) recovering fermentable sugars from the aqueous solution or slurry following the incubation. The fermentable sugars can be recovered by collecting a supernatant, wherein the supernatant comprises the fermentable sugars. The supernatant can be generated by a method selected from the group consisting of: settling; centrifugation; and a combination thereof.

Resulting fermentable sugars can be concentrated by a method selected from the group consisting of: evaporation; spray drying; and a combination thereof.

In yet another particular embodiment described herein is a method for extracting lignin from lignocellulosic plant material, comprising the steps of: a) providing an aqueous solution or slurry comprising lignocellulosic plant material; b) inoculating the aqueous solution or slurry with an effective amount of Thermomyces lanuginosus or one or more derivatives thereof; c) incubating the aqueous solution or slurry with the effective amount of Thermomyces lanuginosus or one or more derivatives thereof; and d) extracting and recovering lignin from the aqueous solution or slurry.

In another particular embodiment described herein is a method for producing a crude enzyme extract for the use in any one of the claims herein, comprising: a) providing lignocellulosic biomass, wherein the lignocellulosic biomass is a biomass of a hardwood plant species comprising terpene resins;

b) submerging the lignocellulosic biomass in an aqueous solution; c) inoculating the lignocellulosic biomass with Thermomyces lanuginosus; d) incubating the lignocellulosic biomass inoculated with Thermomyces lanuginosus; and e) recovering the aqueous solution, wherein the aqueous solution comprises a crude enzyme extract.

### BRIEF DESCRIPTION OF THE DRAWINGS

The patent or application file may contain one or more drawings executed in color and/or one or more photographs. Copies of this patent or patent application publication with color drawing(s) and/or photograph(s) will be provided by the Patent Office upon request and payment of the necessary fee.

FIG. 1: Schematic of fungal enzymes hydrolysis of lignocellulosic biomass.

FIG. 2: Schematic of fermentable sugar production from hydrolysis of cellulose and hemicelluloses.

FIG. 3: Schematic showing bioconversion of sugars to bulk chemicals and fuels.

FIG. 4: Schematic showing specialized applications of fungal enzymes.

FIG. 5A: Schematic showing processes for both fungal enzymatic extraction of rubber from plants and fungal enzymatic biomass hydrolysis.

FIG. 5B: Diagram showing extracellular enzymes produced by Thermomyces lanuginosus grown on guayule bagasse.

FIG. 6A: Bar graph showing enzyme activity of extracellular enzymes produces by Thermomyces lanuginosus grown on wheat straw. Enzyme activities can be found in Table 1 (Example 1).

FIG. 6B: Bar graph showing enzyme activity of extracellular enzymes produces by Thermomyces lanuginosus grown on guayule bagasse. Enzyme activities can be found in Table 1 (Example 1).

FIG. 6C: Bar graph showing results of DNS analysis of sugars produced by hydrolysis with Thermomyces lanuginosus crude enzyme extract. Maximum resulting sugar concentrations can be found in Table 2 (Example 1).

FIGS. 7A-7C: Photographs showing Thermomyces lanuginosus crude enzyme-mediated-purification of natural rubber from TK roots. The photographs show extraction after inulin extraction and filtration.

FIG. 7D: Photograph showing extracted rubber.

FIG. 7E: Photograph showing control rubber extracted by a method based generally on the Eskew process.

FIG. 8: Photograph showing peak activities of extracellular enzymes of Thermomyces lanuginosus (left), and Aspergillus terreus (right).

FIG. 9: Photograph of plant material in aqueous solution during inulin extraction, after the plant materials had been alkaline pretreated.

FIG. 10: Photograph showing rubber extracted by inoculation of TK roots with live Thermomyces lanuginosus.

FIG. 11: Photograph showing rubber extracted by inoculation of TK roots with crude enzyme extract of Thermomyces lanuginosus.

FIGS. 12A-12B: Scanning electron microscope images of alkaline pretreated guayule substrate (FIG. 12A) or wheat straw substrate (FIG. 12B) inoculated with Thermomyces lanuginosus crude enzyme extract.

FIGS. 13A-13B: Scanning electron microscope images of natural rubber extracted from TKS using either the Eskew process (FIG. 13A), or the Thermomyces lanuginosus crude enzyme extract. Images show the enhanced purity of natural rubber extracted using the Thermomyces lanuginosus crude enzyme extract relative to the Eskew process. The natural rubber extracted using the Thermomyces lanuginosus crude enzyme extract is nearly free of impurities.

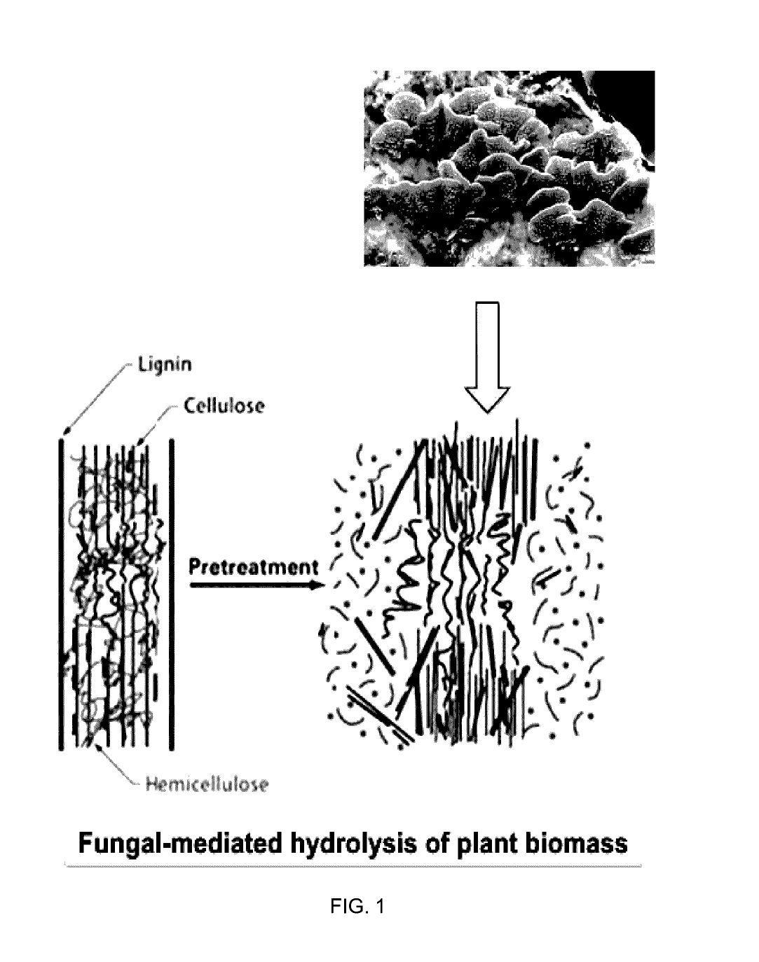

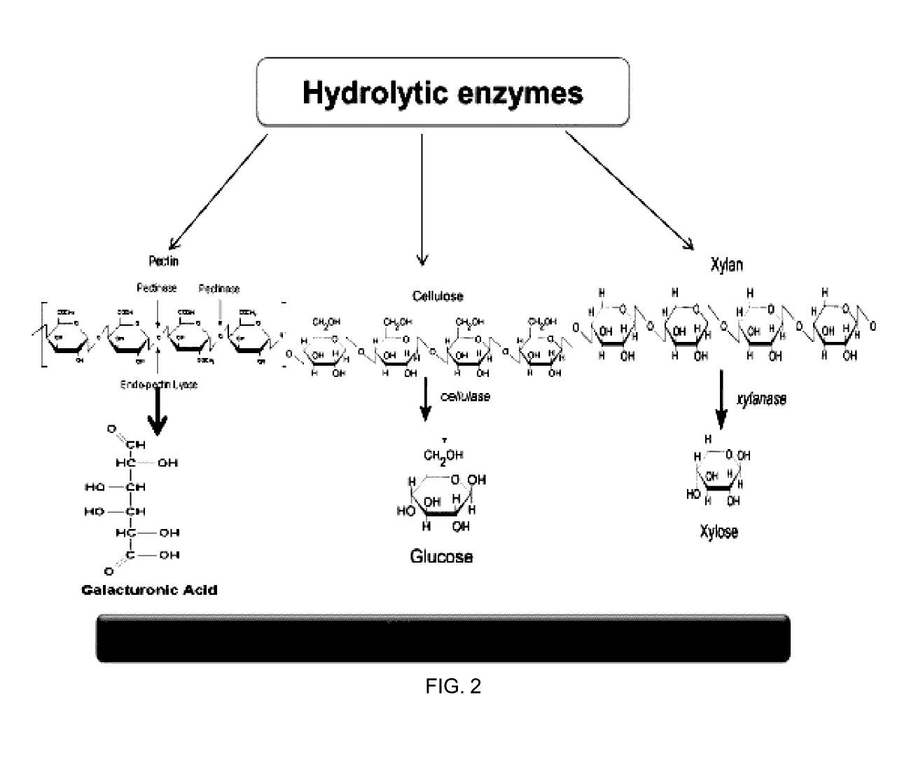

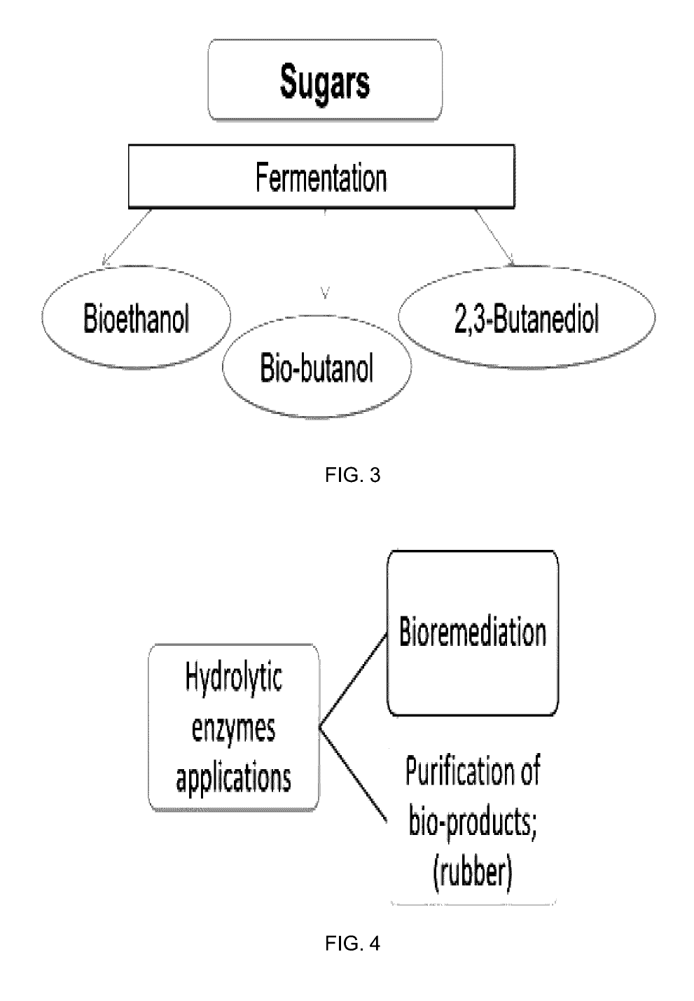

### DETAILED DESCRIPTION

Throughout this disclosure, various publications, patents and published patent specifications are referenced. The disclosures of these publications, patents and published patent specifications are hereby incorporated by reference into the present disclosure to more fully describe the state of the art to which this invention pertains.

Disclosed herein are materials and methods to enhance extractable natural rubber yields from plants. The materials and methods described herein can be used with minimal capital investment, and can provide new agricultural, manufacturing, sales, and transport jobs for local economies. The materials and methods described herein can also be used in conjunction with presently known natural rubber extraction methods to enhance extractable natural rubber yields. Also described herein are methods for producing a crude enzyme extract for use in rubber extraction methods of the present invention.

### DEFINITIONS

As used herein and in the appended claims, the singular forms “a,” “and,” and “the” include plural reference unless the context clearly dictates otherwise.

The term “about” is used herein to mean approximately, roughly, around, or in the region of. When the term “about” is used in conjunction with a numerical range, it modifies that range by extending the boundaries above and below the numerical values set forth.

As used herein, the word “or” means any one member of a particular list and also includes any combination of members of that list.

As used herein, “plant,” “plants,” and/or “plant material” refers to any organism of the kingdom plantae, or part thereof. The terms may refer to the roots, trunk, crown, bark, inner wood, branch(es), leaf (leaves), stem(s) of a plant, or any combination thereof. Therefore, the term may encompass, for example, a portion of a plant comprising a section of the root system remaining attached to a portion of the trunk or stem(s), the bark of a plant alone, a section of trunk or stem(s) attached to one or more branches, one or more branches with attached leaves, an entire plant, etc.

As used herein the term “chop” means reducing plant material to smaller segments. Chopping may be carried out by one or more machines or tools, including but not limited to wood chippers, mulchers, anvil choppers, or other types of choppers.

As used herein, the terms “grind” and “grinding” mean reducing plant material to smaller particles. The particles produced by grinding are smaller than those produced by chopping. Grinding plant material may be carried out by one or more machines or tools, including but not limited to saws, drills, sanders, blenders, pebble mills, hammer mills, ball mills, grist mills, planers, or other types of grinders.

As used herein, the terms “homogenize” and “homogenizing” refer to the process of reducing plant material to particles distributed in a liquid. Generally, homogenization of plant materials occurs in a solution (“homogenization solution”). Homogenization of plant material in homogenization solution may result in a slurry, or homogenate. Filtering the homogenate, thereby removing solid material down to the size of the pores in the specific filter or screen used from the homogenate, results in a “liquid homogenate.”

“Bagasse” is used as is understood in the art: fibrous lignocellulosic biomass of plant material that is insoluble and is suspended rather than dissolved by organic solvents. As used herein, “bagasse” should be understood to include dirt and ash, unless otherwise specified.

### DESCRIPTION

One embodiment of the present invention comprises a method for enhancing extractable rubber yield from plants. Many different rubber-bearing plants and plant materials can be used in the methods disclosed herein, including but not limited to rubber dandelion (Taraxacum kok-saghyz); guayule

(Parthenium argentatum); rubber tree (Hevea brasiliensis), gopher plant (Euphorbia lathyris), mariola (Parthenium incanum), rabbitbrush (Chrysothanmus nauseosus), candelilla (Pedilanthus macrocarpus), Madagascar rubber vine (Cryptostegia grandiflora), milkweeds (Asclepias spp.), goldenrods (Solidago spp.); Scorzonera tau-saghyz; mountain mint (Pycnanthemum incanum), American germander (Teucreum canadense), tall bellflower (Campanula americana), Palaquium gutta; rubber fig (Ficus elastic); Indian banyan (Ficus benghalensis); Panama rubber tree (Castilla elastic); prickly lettuce (Lactuca seriola); crisphead lettuce (Lactuca sativa); pale Indian plantain (Cacalia atriplicifolia); Canada germander (Teucrium canadense); sunflower (Helianthus annus); painted spurge (Euphorbia heterophylla); Euphorbia lactiflua; Euphorbia characias; jackfruit (Artocarpus heterophyllus); plants from the Asteraceae (Compositae), Euphorbiaceae, Campanulaceae, Labiatae, and Moraceae families, and a combination thereof. In a preferred embodiment, the plant is Taraxacum kok-saghyz (TK), and more preferably roots thereof. In other embodiments the plant is Parthenium argentatum (guayule).

TK and guayule are both known natural rubber alternatives to Hevea brasiliensis (Hevea), and were successfully exploited during World War II for the manufacture of tires when access to Asian sources of natural rubber was cut off. However, when access was restored and Hevea plantations were redeveloped, the superior economics of Hevea natural rubber resulted in abandonment of domestic rubber programs in the Soviet Union (TK) and the United States (guayule and TK). Improvements to germplasm were lost for both crops.

Renewed interest in alternatives to Hevea natural rubber has resulted in significant research into both germplasm development and rubber extraction processes. Both TK and guayule produce high molecular weight, high-quality rubber capable of replacing natural rubber from Hevea. While TK and guayule are frontrunners as alternatives to Hevea for natural rubber, it will be recognized that the materials and methods of the present invention can be used to extract rubber from nearly any rubber-bearing plant or plant material.

As described herein, natural rubber can be extracted from TK with enhanced yield and purity relative to other extraction methods. An additional benefit of TK as a source of natural rubber is that TK processing can be optimized to utilize all parts of the crop, as well as by-products. For example, the leaf can be used for salad production or dried and used as a herbal infusion for beverages, or as an animal feed supplement, while the roots can be processed to extract both natural rubber and fermentable sugars. TK roots comprise insoluble fiber (e.g., lignin), soluble sugars, and rubber, and processing can be optimized to extract one, or a combination, of these components. In some embodiments described herein, dried TK roots are processed to extract rubber, fermentable sugars, lignin, or a combination thereof. It will be recognized that the materials and methods described herein can be used to extract any one, or a combination, of these root components.

Each component has an economic value, and, depending on market demand and value, the methods of the present invention can be modified to favor production of a particular component. The rubber component of TK roots can be used in the production of any good currently produced with Hevea natural rubber, including tires. Fermentable sugars, including glucose, xylose, arabinose, and cellobiose, can be used as a carbon source for fermentation to biofuels and platform chemicals. Lignin, an amorphous polymer that acts as a binding agent to provide plants with their structural integrity, can be used in a wide range of applications, including as a binder, a dispersant, and emulsifier, and as a sequestrant.

The rubber extraction methods described herein generally comprise the steps of providing rubber-bearing plant material in aqueous solution or in a slurry, inoculating the aqueous solution or slurry with an effective amount of live Thermomyces lanuginosus or one or more derivatives of the Thermomyces lanuginosus, incubating the aqueous solution or slurry containing the plant matter with the effective amount of Thermomyces lanuginosus or derivative thereof, and recovering natural rubber from the aqueous solution following the incubation step. As used in reference to a method for extracting rubber from a plant, “effective amount” refers to an amount of live Thermomyces or derivative thereof having sufficient enzyme activity to disrupt the linkages between the natural rubber and lignin and other bagasse components.

Wherein the plant material is TK, the roots are preferably separated from the remainder of the plant. As described above, the leaf can be utilized for food, feed, and beverage applications. While whole plants can be used, rubber extraction will be less efficient. Roots are cleaned with water and/or compressed air to remove residual soil and non-root plant matter. Following cleaning, TK roots are dried until the water content is reduced to less than about 30%, and preferably to less than about 10%, thereby coagulating the rubber. The roots can be dried at ambient temperatures or at elevated temperatures of about 60° C. to about 100° C. Dried roots are then stored at low humidity and ambient temperature.

Physical disruption of the plant material is required for efficient rubber extraction. In TK, latex rubber is produced in lactifer storage vessels within the roots, while in guayule, latex rubber is synthesized in specialized parenchyma cells in the plant's bark. This difference in natural rubber synthesis and storage results in different levels of processing of the plant material. Clean, dry TK roots, wherein the latex rubber is coagulated, are processed by chopping, grinding, or homogenization. Preferably, the roots are chopped into particles having a length of about 5 mm to about 20 mm. Wherein the rubber-bearing plant material is guayule, the plant can be similarly chopped. It will be recognized that depending on the location of rubber production and storage in a particular plant, different pre-extraction processing steps may be necessary in order to maximize efficiency of rubber extraction. Determining such processing steps for a particular plant or part of a plant is well within the means of one skilled in the art, and are not limited by those methods described herein.

The chopped, ground, and/or homogenized plant material optionally undergoes additional processing, including but not limited to alkaline pretreatment and boiling. Alkaline pretreatment of lignocellulosic plant material results in fiber reduction and at least partial solubiliztion of organic carbon and proteins. In certain embodiments, plant material is pretreated with an alkaline solution (e.g., NaOH) for 12 to 24 hours at temperatures of about 20° C. to about 55° C. In a particular embodiment, dried TK roots are pretreated with sodium hydroxide (40 g/L) for 24 h at room temperature. Alkaline pretreatment of lignocellulosic biomass is known in the art for biomass processing. Those of skill in the art will recognize that alkaline pretreatment conditions can be adjusted to give a desired result with any particular starting plant material.

Boiling of the plant material functions to extract inulin, a naturally occurring polysaccharide belonging to the fructan class of dietary fibers, as well as other fermentable sugars. Extraction of these carbohydrates is desirable prior to rubber extraction, as it increases rubber extraction efficiency. As described above, the inulin and other sugars can be recovered for use in products such processed foods, or to serve as a carbon source for production of biofuels. In a particular embodiment, the plant material is boiled in aqueous solution one or more times, each boil followed by a filtration step wherein the solid plant material is retained for subsequent boiling and/or processing, and the resulting liquid phase comprising the carbohydrates can be collected for recovery of inulin and other sugars.

Prior to rubber extraction, plant material may undergo alkaline pretreatment, boiling, or any other pretreatment steps to increase efficiency of rubber extraction. These steps can be undertaken in any order, although the order of the pretreatment steps can affect extractable rubber yield and purity. In a preferred embodiment, prior to rubber extraction, plant material is subjected to alkaline pretreatment followed by boiling.

The chopped, ground, and/or homogenized plant material is suspended in an aqueous solution or, where the plant material has been homogenized, in an aqueous slurry. Wherein the plant material has undergone alkaline or boiling pretreatment steps, the plant material can be optionally filtered, separating the solid rubber-bearing plant material from the processing solution. The solid rubber-bearing plant material is then suspended in an aqueous solution or slurry.

The aqueous solution or slurry is inoculated with an effective amount of live Thermomyces lanuginosus, or a derivative thereof. Thermomyces lanuginosus is a widespread thermophilic fungus. All strains of this fungus have been reported to produce extracellular thermostable hemicellulases, as well as other thermostable hydrolytic enzymes. Several strains, for example, have been shown to produce high levels of cellulose-free beta-xylanase. As described herein, live Thermomyces lanuginosus, or derivatives thereof, are used to enhance extractable rubber yield from rubber-bearing plants or plant parts. Effective amounts of live Thermomyces lanuginosus can range between 1.08×10⁶/ml to 2.7×10⁶/ml. The yield of extractable rubber is considered enhanced relative to other known methods wherein the materials of the present invention are used when the yield exceeds that of the other method by 10%, at least 25%, at least 50%, at least 75%, at least 100%, at least 150%, at least 200%, at least 250%, at least 300%, at least 350%, at least 400%, and at least 450%, or at least 500%. In certain embodiments, the yield of extractable rubber is enhanced by at least 100%.

The extracellular enzymes excreted by Thermomyces lanuginosus grown as described herein are capable of enhancing extractable rubber yield in plants. This is likely due to the unique enzyme fingerprint generated by the fungus, wherein the particular combination of excreted enzymes is capable of disrupting the tight linkages (likely covalent) between the bagasse and the rubber, which generally prevent the bound rubber from being extracted. By breaking these linkages, an enhanced yield of extractable rubber is achieved relative to methods wherein Thermomyces lanuginosus or a derivative thereof is not used in the extraction process. For example, incubation of alkaline pretreated and boiled TK roots with either live Thermomyces lanuginosus or a crude enzyme extract thereof enhanced rubber yield by 375% and 320%, respectively, relative to a process based upon the Eskew method of rubber extraction (U.S. Pat. No. 2,393,035), wherein carbohydrates are extracted with hot water prior to mill-crushing TK roots, which liberates the rubber from the bagasse (see Table 3 of Example 2). Live fungus and crude extract also enhanced rubber yield by 200% and 170%, respectively, relative to a process based on the Eskew method that further utilized a combination of commercial enzymes in an effort to release more rubber particles from the bagasse (Table 3 of Example 2).

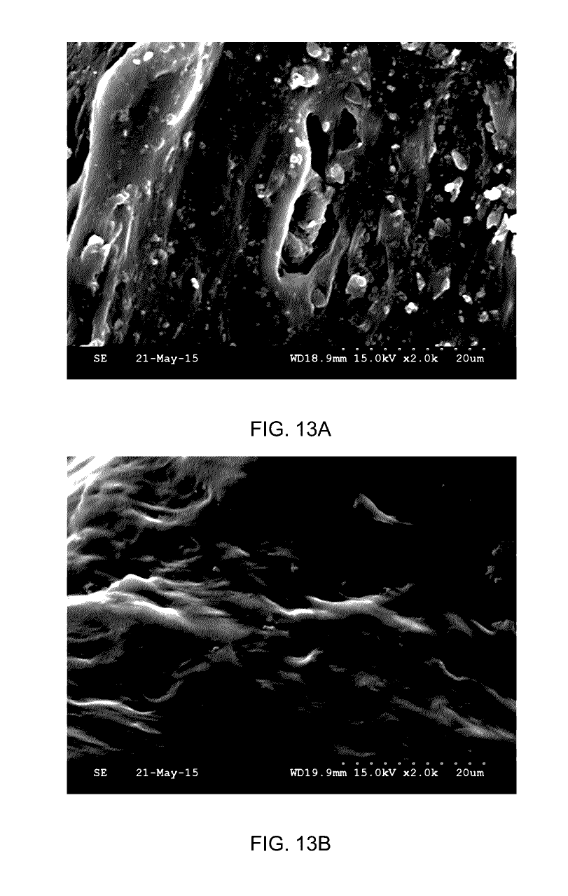

Use of live Thermomyces lanuginosus or a crude enzyme extract thereof also produced high-purity rubber (Table 3 Example 2; FIG. 13). Thus, the methods described herein result in enhanced yield of high purity rubber. In certain embodiments, crude enzyme extracts of Thermomyces lanuginosus are used to yield high purity natural rubbers. Importantly, the natural rubber is not damaged or harmed by the materials and methods of the present invention.

Any strain of Thermomyces lanuginosus can be used in the methods of the present invention. In certain embodiments, a newly isolated strain of Thermomyces lanuginosus is used. The newly isolated strain is Thermomyces lanuginosus isolate STm, the partial genome sequence of which is accessible as GenBank accession number KJ432867.1. The strain is presently being maintained and stored by both Dr. Katrina Cornish at Ohio State University, College of Food, Agriculture, and Environmental Sciences, and Dr. Naeem Ali at Quaid-i-Azam University.

Thermomyces lanuginosus useful for the extraction of rubber as disclosed in the present invention are grown on lignocellulosic biomass. Nearly any lignocellulosic biomass can be used as a substrate for growing Thermomyces lanuginosus. For example, the fungus can be grown on wheat straw,

wheat bran, and guayule bagasse.

Aqueous solutions of lignocellulosic biomass serving as a substrate for Thermomyces lanuginosus are inoculated with the fungus and incubated. The submerged cultures are incubated in conditions conducive to fungal growth, allowing the Thermomyces lanuginosus to produce and excrete extracellular enzymes. Live fungus can then be isolated for use in the methods described herein.

Alternatively, one or more derivatives of the fungus can be used. For example, aqueous samples comprising a crude enzyme extract from the fungus can be collected. Further, enzymes can be purified utilizing methods known in the art. Therefore, the present invention contemplates the use of live Thermomyces lanuginosus, crude enzyme extracts thereof, enzymes or enzyme mixtures wherein the enzymes are purified from the Thermomyces lanuginosus, or any combination thereof. For example crude enzyme extracts can be supplemented with additional purified enzyme of a particular type. Enzyme extracts can also be supplemented with commercially available hydrolytic enzymes, including but not limited to cellulases, xylanases, and hemicellulases. In certain embodiments, submerged cultures containing the selected biomass and the fungus are incubated at 55° C. for 4 to 10 days while shaking. In a preferred embodiment, submerged guayule bagasse in inoculated and the mixture incubated at 55° C. for 7 days while shaking, at which time the aqueous solution containing Thermomyces lanuginosus extracellular enzymes is collected for use as a crude enzyme extract. Depending on desired enzyme activity levels, crude extract samples can be collected at time intervals throughout the incubation period. Longer incubation periods result in higher crude extract enzyme levels.

Thermomyces lanuginosus grown on different substrate results in diverse fungal enzyme fingerprints. For example, while fungal growth is observable on wheat bran, Thermomyces lanuginosus grown on this substrate produces extracellular enzymes having low enzymatic activities (see Table 1 of Example 1). In contrast, Thermomyces lanuginosus grown on substrates of wheat straw or guayule bagasse produces extracellular enzymes having high enzymatic activity (Table 1). The substrate also affects the composition of the fungal enzyme fingerprint, making certain substrates better than others for producing Thermomyces lanuginosus capable of enhancing extractable rubber yield from plants or plant parts. Grown on wheat straw, the live fungus or a crude extract comprising the fungus' extracellular enzymes, for example, produce lower yields of less pure natural rubber than Thermomyces lanuginosus (or a crude extract thereof) grown on a substrate of guayule bagasse.

By growing Thermomyces lanuginosus on guayule bagasse, a plant with a terpene resin content of about 10% normally resistant to disease including fungal growth, the fungus expresses and produces extracellular enzymes that are not expressed when grown on less selective substrates such as wheat bran and wheat straw, which have considerably lower terpene resin content and lower resistance to disease. In certain embodiments, Thermomyces lanuginosus useful in methods for extracting rubber of

the present invention are grown on hardwood plant bagasse substrates with relatively high terpene resin content. Preferably, the terpene resin content of the plant is about 10%. In certain embodiments, the substrate is guayule bagasse, eucalyptus bagasse, or bagasse of another hardwood plant high in terpenes. In a preferred embodiment, the substrate is guayule bagasse.

Following inoculation of the aqueous solution or slurry comprising the rubber-bearing plant material with an effective amount of live Thermomyces lanuginosus or a derivative thereof, as described above, the mixture is incubated. Following the Arrhenius equation, incubation times can be varied, dependant on the incubation temperature. Where lower temperatures are used, longer incubation times will be necessary to allow the extracellular Thermomyces lanuginosus enzymes to disrupt the linkages between the bagasse and the natural rubber. Generally, incubation temperatures can range from about 40° C. to about 90° C. Incubation temperature can be, for example, at least 40° C., at least 45° C., at least 50° C., at least 55° C., at least 60° C., at least 65° C., at least 70° C., at least 75° C., at least 80° C., at least 85° C., or at least 90° C. Incubation times can range from 12 hours to 168 hours. More particularly, incubation times can be, but are not limited to at least 12 hours, at least 24 hours, at least 36 hours, at least 48 hours, at least 72 hours, at least 96 hours, at least 120 hours, at least 144 hours, and at least 168 hours In a preferred embodiment, the mixture is incubated at about 50° for 72 to 96 hours. The incubation temperature and duration can be adjusted to suit any particular plant material. One of skill in the art will recognize that different plants, or even different plant parts of the same plant, will require particular incubation conditions in order to maximize the disruption of the covalent linkages between the natural rubber and the bagasse, and be able to adjust the conditions accordingly. During incubation, the mixture is optionally shaken or stirred.

Following incubation and sufficient disruption of the linkages between the natural rubber and the bagasse, the natural rubber is recovered. Recovery of the natural rubber can be carried out by any method known in the art, including but not limited to flotation (including air flotation), solvent extraction, centrifugation, and combinations thereof. In a preferred embodiment, the natural rubber is recovered by flotation. It will be recognized by those of skill in the art that solution temperature and pH can be adjusted in order to improve flotation efficiency without affecting the quality or purity of the extracted natural rubber. In addition to flotation, other processes, including but not limited to solvent extraction and flocculation can be used to further enhance rubber recovery following Thermomyces lanuginosus enzyme treatment.

The materials and methods for extracting rubber utilizing Thermomyces lanuginosus described herein can also be used in conjunction with current methods, such as those based upon the Eskew process. In any such process, the materials of the present invention—including live Thermomyces lanuginosus, crude enzyme extracts thereof, purified enzymes thereof, or any combination thereof can be added to an aqueous mixture comprising rubber-bearing plant material during or following chopping, grinding, and/or homogenization of the plant material. Preferably, materials of the present invention are added to plant material following carbohydrate (e.g., inulin) extraction.

As described above, rubber extraction processing of rubber-bearing plants results in the creation of several non-rubber by-products, including but not limited to fermentable sugars and lignin. In certain embodiments, fermentable sugars, lignin, or a combination thereof is recovered during the rubber extraction process. In some embodiments, the extraction and recovery of the natural rubber, fermentable sugar, and lignin occurs substantially simultaneously. In a particular embodiment, high-quality lignin is generated by the methods described herein due to the ability of Thermomyces lanuginosus enzymes to disrupt the bonds between the natural rubber and the lignin of the plant bagasse. Both recovered fermentable sugars and lignin can be used for multiple purposes, as described above.

Despite the ability to incorporate the materials and methods of the present invention into currently known natural rubber extraction processes, the materials and methods described herein are advantageous in that they do not require significant capital investment, costly solvents and other materials, the necessary materials are readily produced, and the process is scalable.

Because the methods described herein generally comprise incubating plant material inoculated with materials of the present invention at an elevated temperature, little specialized equipment is required. For example, used fermentation tanks can be used for incubation steps when the process is used on an industrial scale. While certain embodiments may call for specialized equipment, such as a centrifuge or spry dryer, these machines are not required in order to use materials of the present invention according to the methods disclosed herein. The methods of the present invention generally require only water and a hydroxide (e.g., sodium hydroxide), reducing the cost relative to other methods, which may involve expensive chemicals, solvents, and enzymes. Further, the necessary enzymes can be produced-in house, as described above, and the methods are readily scalable from a lab scale, to a pilot plant scale, to an industrial scale.

In a particular embodiment described herein is a method for purifying natural rubber. In this embodiment, a previously extracted and recovered natural rubber is contacted with an effective amount of Thermomyces lanuginosus, or one or more derivatives thereof. These materials are described above. In reference to purification, “effective amount” means an amount of live Thermomyces or derivative having sufficient enzyme activity to dissolve at least a fraction of solid impurities entrained in the natural rubber.

The previously extracted and recovered natural rubber preferably comprises a water content of at least 10% to about 60% to allow the Thermomyces lanuginosus or one or more derivatives thereof to dissolve impurities entrained in the natural rubber. In a particular embodiment, the water content of the natural rubber is at least 10%. In another embodiment, the water content of the natural rubber is at least 50%.

The contacting step can be carried out at ambient temperature, or more preferably, at an elevated temperature of about 40° C. to about 90° C. In a preferred embodiment, the contacting step is carried out at a temperature of about 55° C. The contacting step occurs for a duration of at least 12 hours, at least 24 hours, at least 36 hours, at least 48 hours, at least 72 hours, at least 96 hours, at least 120 hours, at least 144 hours, and at least 168 hours. In a preferred embodiment, the contacting step occurs for a duration of at least 96 hours.

The purification methods described herein can be carried out on natural rubber extracted using any known process. For example, natural rubber extracted utilizing a process based on the Eskew extraction method, further comprising using commercially available enzymes to assist in rubber extraction, was contacted with the materials of the present invention and an additional degree of purity was achieved.

In other embodiments described herein are methods for lignocellulosic biomass saccharification. Unlike the methods of the present invention for extracting rubber from plants, saccharification can be performed on any lignocellulosic biomass. Saccharification methods are similar to method for rubber extraction described above, but avoid any boiling steps. An aqueous solution or slurry comprising lignocellulosic plant material is provided. The plant material is optionally pretreated as described above. The aqueous solution or slurry is then inoculated with an effective amount of Thermomyces lanuginosus or a derivative thereof. In reference to saccharification, an “effective amount” refers to an amount of live Thermomyces or derivative having sufficient enzyme activity to hydrolyze the plant material, and release at least a fraction of the plant materials' fermentable sugars. The inoculated plant material is incubated as described above. Following incubation, fermentable sugars are recovered from the aqueous solution or slurry.

Fermentable sugars can be recovered from the aqueous solution or slurry by any method known in the art. In particular embodiments, a supernatant comprising the fermentable sugars is collected, wherein the supernatant is generated by a methods including but not limited to settling, centrifugation, and a combination thereof. These methods separate the plant material bagasse from the liquid phase of the solution or slurry, wherein the liquid phase comprises the desired sugars.

The recovered sugars can be concentrated into a syrup for use as a carbon source for biofuel production, or any other known use of biomass-derived sugars. The sugars can be concentrated by any means known in the art, including but not limited to evaporation, spray drying, and a combination thereof.

In another embodiment of the present invention, materials described herein are utilized to extract lignin from lignocellulosic plant material. An aqueous solution or slurry comprising lignocellulosic plant material is inoculated and incubated with an effective amount of Thermomyces lanuginosus or one or more derivatives thereof. Optionally, the plant material is pretreated, as described above for the rubber extraction methods. The incubation step occurs similarly that described for the rubber extraction methods above. In reference to a method of extracting lignin from lignocellulosic plant material, an “effective amount” of Thermomyces lanuginosus or a derivative thereof refers to an amount of fungus or fungal enzyme extract sufficient to disrupt the linkages between the lignin and other plant material components, including rubber. Following incubation with materials of the present invention, lignin is sufficiently freed from other plant material components, and can be recovered for downstream use, as described above.

### EXAMPLES

The methods and embodiments described herein are further defined in the following Examples. Certain embodiments of the present invention are defined in the Examples herein. It should be understood that these Examples, while indicating preferred embodiments of the invention, are given by way of illustration only. From the discussion herein and these Examples, one skilled in the art can ascertain the essential characteristics of this invention and without departing from the spirit and scope thereof, can make various changes and modifications of the invention to adapt it to various usages and conditions.

### Example 1

### Production of Hydrolytic Enzymes by Newly Isolated Strain of Thermomyces lanuginosus and Extraction of Fermentable Sugars from Lignocellulosic Biomass

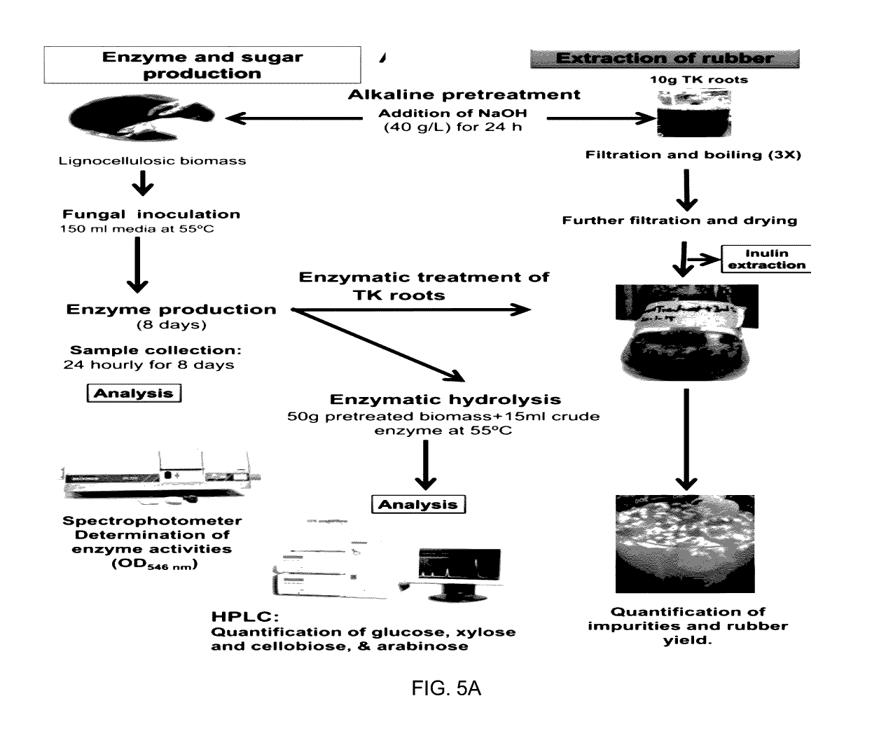

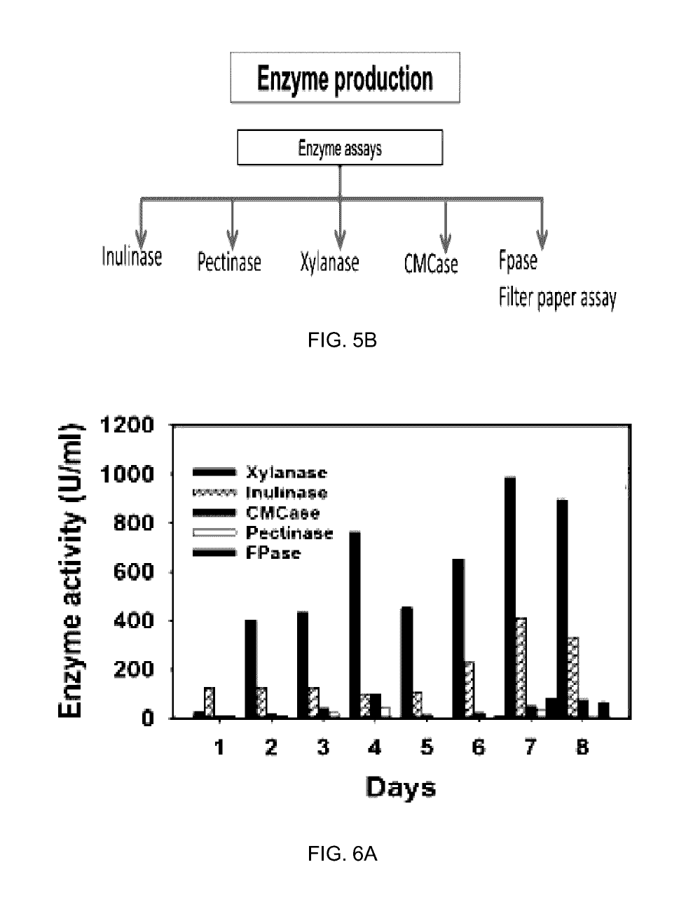

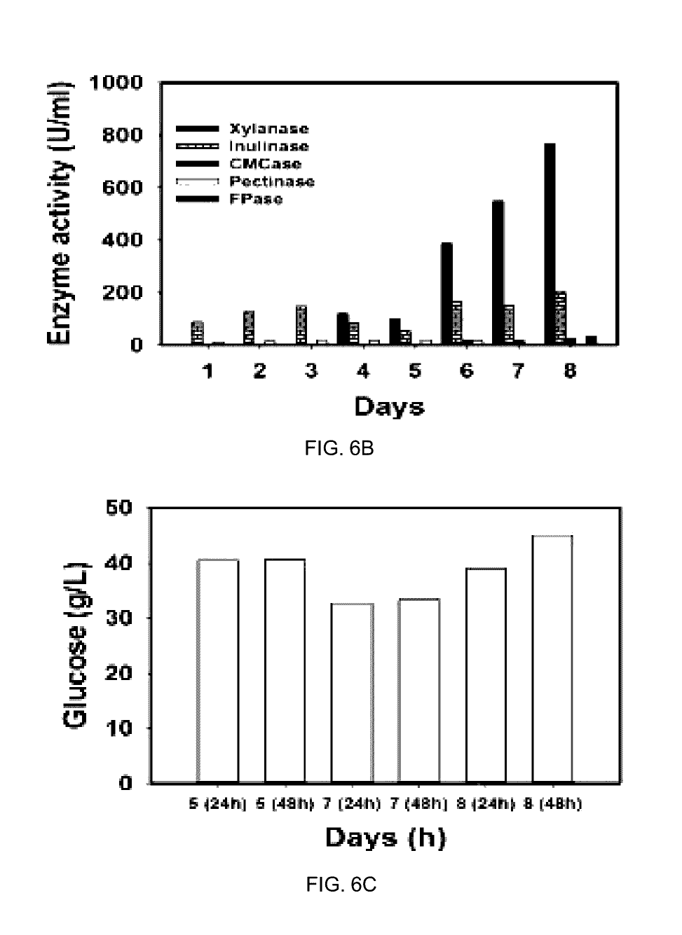

A newly isolated strain of Thermomyces lanuginosus, a thermophilic fungus, was assessed for hydrolytic enzyme activity using three low-cost, alkaline-pretreated lignocellulosic feedstocks: guayule bagasse; wheat bran; and wheat straw. Submerged cultures of the newly isolated T. lanuginosus strain were incubated at 55° C. for 8 days in 150 ml shake flasks containing 4.5 g biomass/l as the carbon source. Considerable amounts of extracellular hydrolytic enzymes were produced (Table 1; FIGS. 5, 6A-6B).

TABLE 1
Maximum enzyme activity during first 7 days of
incubation of newly isolated T. lanuginosus strain
incubated on various carbon sources.

| | Maximum Enzyme Activity During First 7 Days (U ml⁻¹) | | | | |
|---|---|---|---|---|---|
| | | | | Filter Paper | |
| | | | | Assay | |
| | Xylanase | Inulinase | CMCase | (FPase) | Pectinase |
| Wheat | 985.3 | 411 | 98.4 | 83.6 | 42.5 |
| Straw | | | | | |
| Guayule | 766.5 | 201.2 | 18.2 | 115.5 | 18.9 |
| Bagasse | | | | | |
| Wheat | Low | Low | Low | Low | Low |
| Bran | activity | activity | activity | activity | activity |

5 g of pretreated guayule bagasse was inoculated with the newly isolated T. lanuginosus strain, and incubated for 5 to 7 days at 50° C. During incubation, the fungus produced extracellular pectinase, xylanase, inulinases, and cellulases. The extracellular enzymes were collected in aqueous solution as a crude enzyme extract of the newly isolated T. lanuginosus strain. 50 g of alkaline pretreated biomass was inoculated with 15 ml of the crude enzyme extract and incubated for 96 hrs at 55° C.

HPLC analysis detected glucose and cellobiose as the main hydrolytic products, while also detecting xylose and arabinose. Maximum resulting sugar concentrations can be found in Table 2.

Glucose levels were further analyzed by DNS assay (FIG. 6C).

TABLE 2
Levels of fermentable sugars determined by HPLC.

| | Sugar Concentration (g/l) | | | | | |
|---|---|---|---|---|---|---|
| | Day 5 | Day 7 | Day 8 | Day 5 | Day 7 | Day 8 |
| | (24 hr) | (24 hr) | (24 hr) | (48 hr) | (48 hr) | (48 hr) |
| Glucose | 5.5 | 4.74 | 5.9 | 5.48 | 4.65 | 5.91 |
| Xylose | 35.2 | 30.1 | 39.52 | 37.43 | 29.4 | 42.62 |
| Arabinose | 4.27 | 4.04 | 4.85 | 4.55 | 3.97 | 5 |
| Cellobiose | 35.315 | 32.66 | 33.325 | 27.841 | 32.452 | 28.87 |

Extracellular enzymes released by the newly isolated T. lanuginosus strain when grown on guayule bagasse (following latex extraction), wheat straw, or wheat bran were collected in aqueous solution for use as a crude T. lanuginosus enzyme extract (FIG. 5).

### Example 2

### Extraction of Natural Rubber from Roots of Taraxacum kok-saghyz

The newly isolated T. lanuginosus strain was further assessed for its ability to enhance the yield of natural rubber from natural rubber-bearing plant material (FIG. 5).

5 g of pretreated guayule bagasse was inoculated with the newly isolated T. lanuginosus strain, and incubated for 5 to 7 days at 50° C. During incubation, the fungus produced extracellular pectinase, xylanase, inulinases, and cellulases. The extracellular enzymes were collected in aqueous solution as a crude extract of the newly isolated T. lanuginosus strain.

5 or 10 g of chopped Taraxacum kok-saghyz (TKS) roots were pretreated using an alkaline pretreatment (NaOH; 40 g/L), and subsequently boiled three to five time for about 30 minutes each time to extract inulin. The resulting aqueous solution comprising the TKS roots was filtered following each boil.

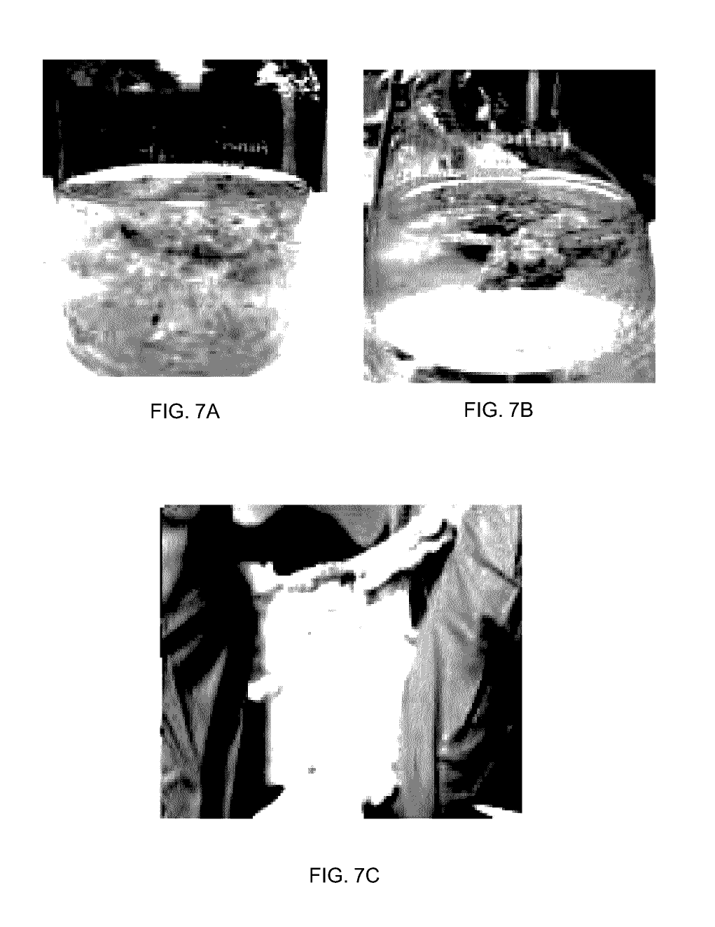

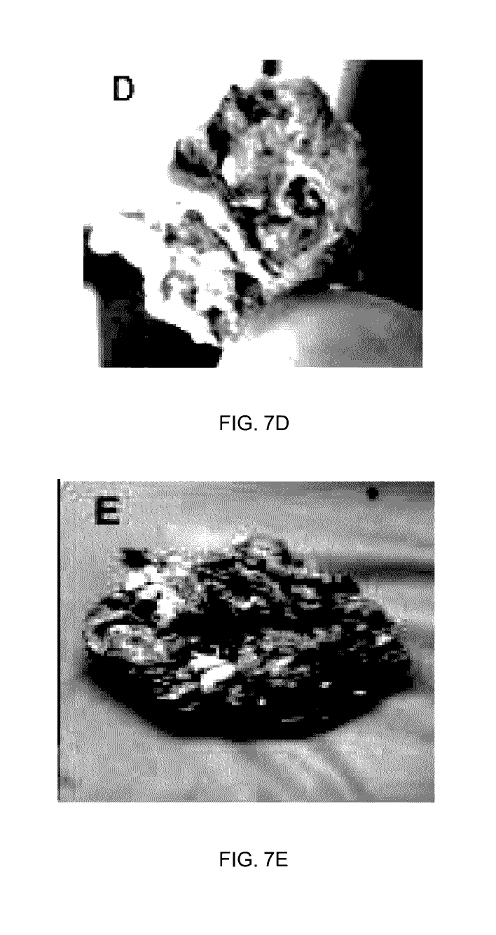

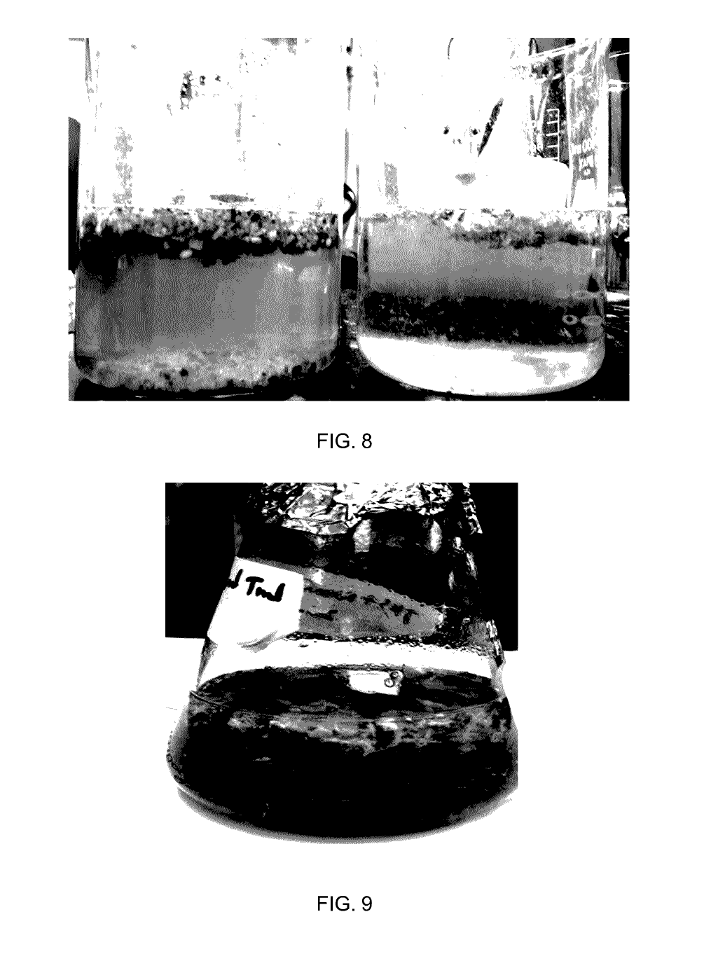

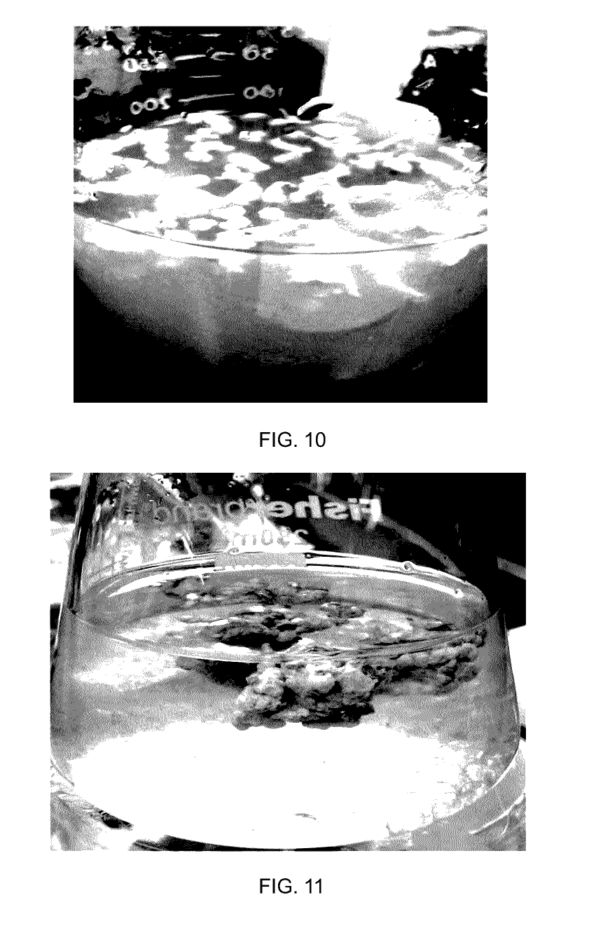

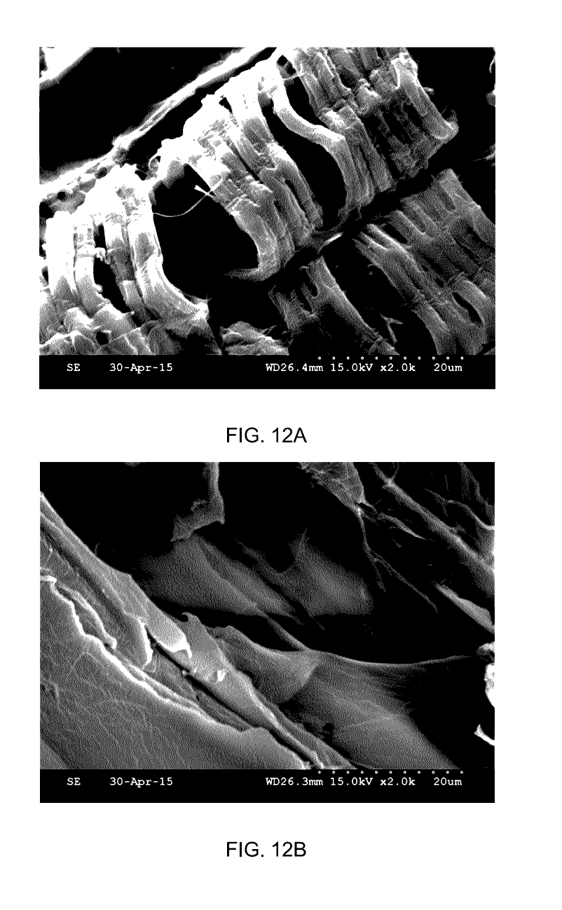

In parallel, alkaline-pretreated roots of Taraxacum kok-saghyz (TK) were inoculated with either live T. lanuginosus or the crude enzyme extract and incubated for 4 days at 50° C. (FIGS. 7-11). Following the incubation period, natural rubber extracted from the TKS roots was recovered and dried at 37° C. for 24 h and weighed (FIGS. 7C-7D). Rubber impurities were analyzed using standard procedures.

T. lanuginosus treatment yielded 90 mg pure rubber/g dry TKS root, which was much higher than the yield of the original process (Eskew method; 24 mg/g), and an improved process utilizing a commercial enzyme combination (45 mg/g). Treatment with the T. lanuginosus crude extract yielded 77 mg pure rubber/g dry TKS root. Treatment with either live T. lanuginosus or the crude enzyme extract also affected rubber purity (Table 3). Further treatment of rubber extracted by the commercial enzyme combination with the crude T. lanuginosus enzyme extract resulted in additional purification of the rubber by dissolving at least a fraction of the entrained solids.

TABLE 3
Rubber yield and purity.

| | Rubber Yield | |
|---|---|---|
| | (mg pure rubber/g | Rubber |
| Extraction Method | dry TKS root) | Purity |
| Original Process | 24 | 37.5 |
| Commercial Enzyme | 45 | 94.1 |
| Combination | | |
| Live T. lanuginosus | 90 | 71.7 |
| Crude T. lanuginosus | 77 | 91.6 |
| enzyme extract | | |

Applicant will deposit the Thermomyces lanuginosus strain STm of the invention with the American Type Culture Collection (ATCC), Manassas, Va., in compliance with the Budapest Treaty and in compliance with 37 C.F.R. §1.801-§1.809. The ATCC Accession No. will be provided upon receipt thereof. Following deposit with the ATCC, access to this deposit will be available during the pendency of this application to persons determined by the Commissioner of Patents and Trademarks to be entitled thereto under 37 CFR §1.14 and 35 USC §122.

While the invention has been described with reference to various and preferred embodiments, it should be understood by those skilled in the art that various changes may be made and equivalents may be substituted for elements thereof without departing from the essential scope of the invention. In addition, many modifications may be made to adapt a particular situation or material to the teachings of the invention without departing from the essential scope thereof.

Therefore, it is intended that the invention not be limited to the particular embodiments disclosed herein contemplated for carrying out this invention, but that the invention will include all embodiments falling within the scope of the claims.

What is claimed is:

1. A method for enhancing yield of natural rubber from plant material, comprising the steps of:
a) providing an aqueous solution or slurry comprising plant material, wherein the plant material comprises natural rubber;
b) inoculating the aqueous solution or slurry with an effective amount of live Thermomyces lanuginosus or one or more derivatives thereof;
c) incubating the aqueous solution or slurry with the effective amount of live Thermomyces lanuginosus or one or more derivatives thereof; and
d) recovering natural rubber from the aqueous solution or slurry following the incubation.

2. The method of claim 1, wherein the plant material is chopped, ground, homogenized, or a combination thereof.

3. The method of claim 1, wherein the plant material is subjected to an alkaline pretreatment prior to the inoculation.

4. The method of any one of claims 1-4, wherein inulin is extracted from the plant material prior to the inoculation.

5. The method of claim 5, wherein the inulin is extracted by boiling the aqueous solution or slurry comprising the plant material one or more times.

6. The method of claim 5, wherein the plant material is filtered following each of the one or more times the aqueous solution or slurry comprising the plant material is boiled.

7. The method of claim 1, wherein the plant material is a plant or a part of a plant selected from the group of plants consisting of: Rubber dandelion (Taraxacum kok-saghyz); guayule (Parthenium argentatum); rubber tree (Hevea brasiliensis); gopher plant (Euphorbia lathyris); mariola (Parthenium incanum); rabbitbrush (Chrysothanmus nauseosus); candelilla (Pedilanthus macrocarpus); Madagascar rubber vine (Cryptostegia grandiflora); milkweeds (Asclepias spp.); goldenrods (Solidago spp.); Scorzonera tau-saghyz; mountain mint (Pycnanthemum incanum); American germander (Teucreum canadense); tall bellflower (Campanula americana); Palaquium gutta; rubber fig (Ficus elastic); Indian banyan (Ficus benghalensis); Panama rubber tree (Castilla elastic); prickly lettuce (Lactuca seriola); crisphead lettuce (Lactuca sativa); pale Indian plantain (Cacalia atriplicifolia); Canada germander (Teucrium canadense); sunflower (Helianthus annus); painted spurge (Euphorbia heterophylla); Euphorbia lactiflua; Euphorbia characias; jackfruit (Artocarpus heterophyllus); and a combination thereof.

8. The method of claim 1, wherein the plant material is a plant or a root of Taraxacum kok-saghyz.

9. The method of claim 1, wherein the effective amount is an amount of live Thermomyces lanuginosus or one or more derivatives thereof capable of disrupting bonds between a bagasse fraction of the plant material and a natural rubber fraction of the plant material, thereby enhancing yield of natural rubber from the plant material relative to a method wherein live Thermomyces lanuginosus or one or more derivatives thereof are not employed.

10. The method of claim 9, wherein yield of natural rubber from the plant material is enhanced by a percentage relative to a method wherein live Thermomyces lanuginosus or one or more derivatives thereof are not employed selected from the group consisting of: at least 10%; at least 25%; at least 50%; at least 75%; at least 100%; at least 150%; at least 200%; at least 250%; at least 300%; at least 350%; at least 400%; and at least 450%; and at least 500%.

11. The method of any one of claims 1-10, wherein the one or more derivatives of the live Thermomyces lanuginosus comprises a crude enzyme extract, one or more purified enzymes isolated from a crude enzyme extract, or a combination thereof.

12. The method of any one of claims 1-11, wherein the live Thermomyces lanuginosus is Thermomyces lanuginosus isolate STm, the partial genome sequence of which is accessible as GenBank accession number KJ432867.1.

13. The method of claim 1, wherein the incubation occurs at a temperature selected from the group of temperatures consisting of: at least 40° C.; at least 45° C.; at least 50° C.; at least 55° C.; at least 60° C.; at least 65° C.; at least 70° C.; at least 75° C.; at least 80° C.; at least 85° C.; and at least 90° C.

14. The method of claim 1, wherein the incubation occurs at a temperature of about 40° C. to about 90° C.

15. The method of any one of claim 1, 13, or 14, wherein the incubation occurs for a duration selected from the group consisting of: at least 12 hours; at least 24 hours; at least 36 hours; at least 48 hours; at least 72 hours; at least 96 hours; at least 120 hours; at least 144 hours; and at least 168 hours.

16. The method of claim 1, wherein the natural rubber is recovered by a method selected from the group consisting of: flotation and skimming; solvent extraction; centrifugation; and a combination thereof.

17. The method of claim 1, further comprising recovering fermentable sugars, lignin, or a combination thereof from the aqueous solution or slurry.

18. A method for purifying natural rubber comprising the steps of:
a) providing a natural rubber; and
b) contacting the natural rubber with an effective amount of Thermomyces lanuginosus or one or more derivatives thereof, thereby purifying the natural rubber.

19. The method of claim 18, wherein the natural rubber is provided in an aqueous solution, wherein the rubber has a water content selected from the group consisting of: at least 10%; at least 20%; at least 30%; at least 40%; at least 50%; and about 60%.

20. The method of claim 18 wherein the natural rubber had previously been purified by a method other than that of claim 18.

21. The method of claim 18 or claim 20, wherein the effective amount is an amount of live Thermomyces lanuginosus or one or more derivatives thereof capable dissolving at least a fraction of solids entrained in the natural rubber.

22. The method of claim 18, wherein the one or more derivatives of the live Thermomyces lanuginosus comprises a crude enzyme extract, one or more purified enzymes isolated from a crude enzyme extract, or a combination thereof.

23. The method of claim 18, wherein the live Thermomyces lanuginosus is Thermomyces lanuginosus isolate STm, the partial genome sequence of which is accessible as GenBank accession number KJ432867.1.

24. The method of claim 18, wherein the contacting step occurs for a duration selected from the group consisting of: at least 12 hours; at least 24 hours; at least 36 hours; at least 48 hours; at least 72 hours; at least 96 hours; at least 120 hours; at least 144 hours; and at least 168 hours.

25. The method of claim 18, wherein the contacting step occurs at a temperature of about 40° C. to about 90° C.

26. The method of claim 18, wherein the contacting step occurs at a temperature of about 50° C.

27. A method for lignocellulosic biomass saccharification comprising the steps of:
a) providing an aqueous solution or slurry comprising lignocellulosic plant material;
b) inoculating the aqueous solution or slurry with an effective amount of Thermomyces lanuginosus or one or more derivatives thereof;
c) incubating the aqueous solution or slurry with the effective amount of Thermomyces lanuginosus or one or more derivatives thereof; and
d) recovering fermentable sugars from the aqueous solution or slurry following the incubation.

28. The method of claim 27, wherein the plant material is ground, chopped, milled, homogenized, or a combination thereof.

29. The method of claim 28, wherein the plant material is filtered.

30. The method of claim 27, wherein the plant material is subjected to an alkaline pretreatment prior to the inoculation.

31. The method of claim 27, wherein the one or more derivatives of the live Thermomyces lanuginosus comprises a crude enzyme extract, one or more purified enzymes isolated from a crude enzyme extract, or a combination thereof.

32. The method of claim 27, wherein the live Thermomyces lanuginosus is Thermomyces lanuginosus isolate STm, the partial genome sequence of which is accessible as GenBank accession number KJ432867.1.

33. The method of claim 27, wherein the incubation occurs at a temperature selected from the group of temperatures consisting of: at least 40° C.; at least 45° C.; at least 50° C.; at least 55° C.; at least 60° C.; at least 65° C.; at least 70° C.; at least 75° C.; at least 80° C.; at least 85° C.; and at least 90° C.

34. The method of claim 27, wherein the incubation occurs at a temperature of about 40° C. to about 90° C.

35. The method of claim 27, wherein the incubation occurs for a duration selected from the group consisting of: at least 12 hours; at least 24 hours; at least 36 hours; at least 48 hours; at least 72 hours; at least 96 hours; at least 120 hours; at least 144 hours; and at least 168 hours.

36. The method of claim 27, wherein the fermentable sugars are recovered by collecting a supernatant, wherein the supernatant comprises the fermentable sugars.

37. The method of claim 36, wherein the supernatant is generated by a method selected from the group consisting of: settling; centrifugation; and a combination thereof.

38. The method of claim 36 or 37, wherein the fermentable sugars are concentrated by a method selected from the group consisting of: evaporation; spray drying; and a combination thereof.

39. A method for lignocellulosic biomass saccharification and enhancing yield of natural rubber from plant materials, comprising the steps of:
a) providing an aqueous solution or slurry comprising lignocellulosic plant material, wherein the lignocellulosic plant material comprises natural rubber;
b) inoculating the aqueous solution or slurry with an effective amount of Thermomyces lanuginosus or one or more derivatives thereof;
c) incubating the aqueous solution or slurry with the effective amount of Thermomyces lanuginosus or one or more derivatives thereof; and
d) extracting and recovering natural rubber and fermentable sugars substantially simultaneously from the aqueous solution or slurry following the incubation.

40. The method of claim 39, further comprising recovering lignin from the aqueous solution or slurry.

41. A method for extracting lignin from lignocellulosic plant material, comprising the steps of:
a) providing an aqueous solution or slurry comprising lignocellulosic plant material;
b) inoculating the aqueous solution or slurry with an effective amount of Thermomyces lanuginosus or one or more derivatives thereof;
c) incubating the aqueous solution or slurry with the effective amount of Thermomyces lanuginosus or one or more derivatives thereof; and
d) extracting and recovering lignin from the aqueous solution or slurry.

42. A method for producing a crude enzyme extract for the use in any one of the claims herein, comprising:
a) providing lignocellulosic biomass, wherein the lignocellulosic biomass is a biomass of a hardwood plant species comprising terpene resins;
b) submerging the lignocellulosic biomass in an aqueous solution;
c) inoculating the lignocellulosic biomass with Thermomyces lanuginosus;
d) incubating the lignocellulosic biomass inoculated with Thermomyces lanuginosus; and
e) recovering the aqueous solution, wherein the aqueous solution comprises a crude enzyme extract.

43. The method of claim 42, wherein the Thermomyces lanuginosus is Thermomyces lanuginosus isolate STm, the partial genome sequence of which is accessible as GenBank accession number KJ432867.1.

44. The method of claim 42, wherein the lignocellulosic biomass is biomass of a plant selected from the group consisting of: guayule (Parthenium argentatum); and Eucalyptus spp.

45. The method of claim 42, wherein the lignocellulosic biomass is biomass of guayule (Parthenium argentatum).

46. The method of claim 42, wherein the incubation occurs for a duration selected from the group consisting of: at least 4 days; at least 5 days; at least 6 days; at least 7 days; at least 8 days; at least 9 days; and at least 10 days.

47. The method of claim 42, wherein the lignocellulosic occurs at a temperature selected from the group of temperatures consisting of: at least 30° C.; at least 35° C.; at least 40° C.; at least 45° C.;
at least 50° C.; at least 55° C.; at least 60° C.; at least 65° C.; and at least 70° C.

48. The method of claim 42, wherein the incubation occurs at a temperature of about 55° C.

49. A crude enzyme extract produced by the method of claim 42.

50. A crude enzyme extract comprising extracellular enzymes from Thermomyces lanuginosus.

51. The crude enzyme extract of claim 50, wherein the Thermomyces lanuginosus is Thermomyces lanuginosus isolate STm, the partial genome sequence of which is accessible as GenBank accession number KJ432867.1.

52. The invention as disclosed and described herein.
